(19) United States
(12) Patent Application Publication
(10) Pub. No.: US 2018/0177703 A1
(43) Pub. Date: Jun. 28, 2018

(54) Niacinamide Mononucleotide Formulations For Skin Aging

(71) Applicant: N.V. Perricone LLC, San Francisco, CA (US)

(72) Inventor: Nicholas V. Perricone, Meriden, CT (US)

(21) Appl. No.: 15/739,219

(22) PCT Filed: Jun. 24, 2016
(86) PCT No.: PCT/US16/39183

§ 371 Date: Dec. 22, 2017

Related U.S. Application Data
(60) Provisional application No. 62/184,634, filed on Jun. 25, 2015.

(51) Int. Cl. A61K 8/60; A61Q 19/08; A61K 8/67; A61K 8/55; A61K 8/37; A61K 8/44; A61K 8/365

(52) U.S. Cl. CPC A61K 8/606; A61Q 19/08; A61K 8/678; A61K 8/365; A61K 8/37; A61K 8/44; A61K 8/55

(57) ABSTRACT

Topical compositions including niacinamide mononucleotide to improve the appearance of aging skin and to prevent and treat skin aging; and methods of use of topical compositions including niacinamide mononucleotide to improve the appearance of aging skin and to prevent and treat skin aging

### FIELD OF THE INVENTION

The present invention relates to topical compositions comprising niacinamide mononucleotide and/or its derivative for skin aging and methods of use of the topical compositions to prevent and treat skin aging.

### BACKGROUND OF THE INVENTION

Human skin is constantly directly exposed to the air, solar radiation, environmental pollutants, or other mechanical and chemical insults, which are capable of inducing the generation of free radicals as well as reactive oxygen species (ROS) of our own metabolism. Extrinsic skin damage develops due to several factors: ionizing radiation, severe physical and psychological stress, alcohol intake, poor nutrition, overeating, environmental pollution, and exposure to UV radiation (UVR). It is estimated that among all these environmental factors, UVR contributes up to 80%. UV-induced generation of ROS in the skin develops oxidative stress, when their formation exceeds the antioxidant defiance ability of the target cell. The primary mechanism by which UVR initiates molecular responses in human skin is via photochemical generation of ROS, mainly formation of superoxide anion (O(2)(−)(.)), hydrogen peroxide (H(2)O(2)), hydroxyl radical (OH(.)), and singlet oxygen ((1)O(2)). Over time, the presence of ROS will cause conditions of aging skin.

Niacinamide, also known as nicotinamide or nicotinic amide, is the amide of nicotinic acid (a.k.a. niacin) and a source of vitamin B₃. Niacinamide can be found in Vitamin B₃ containing foods including yeast, meat, fish, milk, eggs, green vegetables, beans, and cereal grains. Niacin and niacinamide are also found in many vitamin B complex supplements with other B vitamins. In cells, niacinamide is incorporated into coenzymes of nicotinamide adenine dinucleotide (NAD) and nicotinamide adenine dinucleotide phosphate (NADP), which are responsible for a wide variety of enzymatic oxidation-reduction reactions (“redox reaction”). It is believed that niacinamide exerts antioxidant properties through the redox reaction; and that it can scavenge reactive oxygen species. Moreover, it is reported that nicotinamide has demonstrated anti-inflammatory activity.

U.S. Pat. No. 7,179,477 to Gupta, S. K. discloses a three-step dermabrasion system for skin. Niacinamide salts such as niacinamide lactate, niacinamide glycolate, niacinamide malate, niacinamide mandelate, niacinamide ascorbate, niacinamide phytate, niacinamide citrate, niacinamide hydroxycitrate, niacinamide aleurate, niacinamide salicylate, and/or niacinamide hyaluronate are used as skin softening agents in the system.

U.S. Pat. No. 7,320,797 to Gupta, S. K. discloses an anti-aging topical composition for skin comprising (i) a quaternary ammonium extra-cellular antioxidant agent, (ii) an intracellular antioxidant agent, (iii) an antiinflammatory agent, and (iv) a collagen boosting agent, and (v) a carrier base. The collagen boosting agent may include, among many other choices, niacinamide or a niacinamide salt selected from niacinamide lipoate and niacinamide scorbate.

U.S. Application Publication No. 20150093346 to Burke-Colvin, D. discloses skin care formulations in which niacinamide lactate is used as a bleaching and lightening agent.

U.S. Pat. No. 7,700,076 to Tamarkin, D. relates to a pharmaceutical foam composition having enhanced skin penetration for treatment of skin disorders, such as aging skin, wrinkles, hyperpigmentation, scaly skin and other undesirable skin properties. Niacinamide and niacinamide N-oxide can be included in the composition as anti-wrinkle actives and sources of vitamin B₃.

U.S. Pat. No. 8,106,184 to Sauve, A. A. is directed to methods of using nicotinoyl riboside and nicotinamide riboside to increase levels of NAD in cells and tissues for improving their lifespan. It proposes that skin aging can be prevented by treating skin or epithelial cells in accordance with the methods. But no written description of a nicotinoyl riboside or nicotinamide riboside formulation having the proposed effect is disclosed.

WO2015066382 to Deren-Lewis, A., assigned to Chromadex Inc., discloses the use of nicotinoyl riboside or salts thereof for treating signs or symptoms of aging or skin wrinkles in an individual. Chromadex Inc. has a commercial anti-aging supplement sold under the trade name NIAGEN™, which is a chloride salt of nicotinamide riboside supplied in vegetarian capsules for oral administration.

The prior art has disclosed specific nicotinamide derivatives for skin care, namely, niacinamide salts, niacinamide N-oxide, and nicotinamide riboside. In most cases, the nicotinamide derivatives, such as niacinamide salts and niacinamide N-oxide, are merely used as optional adjunct supplements in topical cosmetic compositions. Nicotinoyl riboside or nicotinamide riboside have been proposed, but workable formulations containing nicotinoyl riboside or nicotinamide riboside for topical treatment or prevention of skin aging have not been disclosed.

### SUMMARY OF THE INVENTION

The present invention provides a topical composition for application to aging skin, comprising an effective amount of β-niacinamide mononucleotide (in short, “niacinamide mononucleotide” or “NMN”) and/or its derivative, as shown in formula (I):

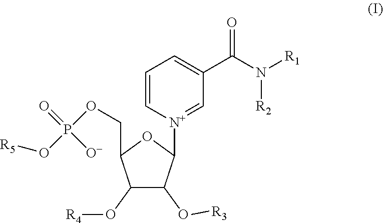

wherein each of R₁ and R₂ is individually selected from the group consisting of hydrogen, alkyl, cycloalkyl, alkenyl, alkaryl, hydroxy, alkoxy, and amino; each of R₃ and R₄ is individually selected from the group consisting of hydrogen, alkyl, aliphatic or aromatic acyl, and aliphatic or aromatic thioacyl; and R₅ is hydrogen, alkyl, or cation. When R₅ is hydrogen, the formula (I) may be in an ionic salt form when a sufficient amount of base is present (not shown). For example, the phosphate acid may be in a sodium salt form, wherein the covalent bond “O—R₅” may be replaced by an ionic bond, “O⁻Na⁺”. In other words, R₅ is a sodium cation. In preferred embodiments, R₁ to R₅ are all hydrogen, in which case formula (I) represents niacinamide mononucleotide.

The topical composition further provides a pharmaceutically acceptable carrier. In some of these embodiments, the niacinamide mononucleotide or its derivative is present from about 0.005% to about 30.0% by weight. In certain of these embodiments, the niacinamide mononucleotide or its derivative is present from about 0.1% to about 20.0% by weight. In certain embodiments, the niacinamide mononucleotide or its derivative is present from about 0.1% to about 5.0% by weight. In further embodiments, the niacinamide mononucleotide or its derivative is present from about 0.5% to about 1.5% by weight.

In some embodiments, the composition further comprises one or more adjunct active ingredients selected from the group consisting of fatty acid, fatty acid ester of ascorbic acid, and the mixture thereof. In some embodiments, the composition further comprises at least one adjunct active ingredient selected from the group consisting of salts of magnesium, zinc and copper, tocotrienols, tocotrienol derivatives, vitamin E compositions enriched with tocotrienols or tocotrienol derivatives (e.g., tocotrienol acetate, also known as vitamin E acetate), and mixtures thereof. In some embodiments, the adjunct active ingredient of the composition comprises a mixture of magnesium aspartate, zinc gluconate and copper gluconate. The adjunct active ingredients may be present from about 0.01% to about 20.0% by weight.

In certain embodiments, the composition comprises an oil-in-water emulsion. In other embodiments, the composition comprises a cream. In the oil-in-water emulsion embodiments, the composition comprises an emulsifier such as fatty acid derivatives of stearic acid or phosphatidylcholine.

As a non-limiting example, the invention provides a topical composition for application to aging skin in the form of a cream or lotion which comprises about 0.1% to about 5.0% by weight, preferably about 0.1% to about 5.0% by weight, and even more preferably about 0.5% to about 1.5% by weight of niacinamide mononucleotide or its derivative, about 2.0% to about 15.0% by weight of at least one emulsifier, and water. In a preferred embodiment, the above topical composition may have at least one adjunct active component in an amount of about 1.0% to about 5.0% by weight of the composition.

The topical composition for application to aging skin further comprises an emollient. One preferred emollient is isopropyl palmitate. In some of these embodiments, the composition comprises about 1.0% to about 3.0% by weight of niacinamide mononucleotide or its derivative, about 1.5% to about 5.0% by weight of at least one fatty acid derivative of stearic acid, about 1.0% to 5.0% by weight of an emollient, and water.

As another example, the invention provides a topical composition for application to aging skin in a gel form which comprises about 10.0% to about 30.0% by weight, preferably about 15.0% to about 25.0% by weight, and even more preferably about 15.0% to about 20.0% by weight of niacinamide mononucleotide or its derivative, about 30.0% to about 65.0% by weight of at least one emulsifier as a gel base. In a preferred embodiment, the gel form topical composition comprises about 15.0% to about 20.0% by weight of niacinamide mononucleotide or its derivative, about 75.0% to about 85.0% of emolliments and/or emulsifiers, and no water.

The topical composition of the invention may contain additional ingredients commonly found in skin care compositions and cosmetics, such as, for example, tinting agents, skin conditioning agents, humectants, preservatives, antioxidants, perfumes, chelating agents.

The present invention further provides a method of topical use of compositions comprising niacinamide mononucleotide and/or its derivative of formula (I) to treat or prevent skin aging. The method comprises the step of topically applying the topical composition to the skin areas at predetermined intervals.

It is noticed that the use of the niacinamide mononucleotide and/or its derivative containing compositions improves the appearance of aging skin, including surface spots, brown spots, red areas, wrinkles and texture and other artifacts of aging skin, as well as conditions of skin dryness, dullness, loss of elasticity, lack of radiance, exaggerated lines and wrinkles, spider vessels or red blotchiness. The appearance of marionette lines, smile lines, deep nasolabial fold lines, crow's feet, fine lines/wrinkles, vertical lines between the eyebrows, horizontal forehead lines, sagging thin/frail skin, skin redness and dullness are improved using the methods of the invention. Thus, the present invention also provides a method of treating aged skin.

### DETAILED DESCRIPTION OF THE INVENTION

Aging skin is characterized histologically by cross-linking of collagen and elastin in the dermis. This results in loss of support seen clinically as sagging and wrinkling. The present invention recognizes these processes and provides compositions and methods to minimize both prospective and existing aging conditions. In particular, the present invention provides topical compositions comprising nicotinamide mononucleotide (NMN) and/or its derivative, when topically applied to skin, such that the rate of regeneration of skin cell tissues is predominant over the rate of degeneration, thereby preventing skin aging conditions.

The term “topical composition” as used herein shall mean the complete product including the niacinamide mononucleotide or its derivative active ingredient, the carrier, and any adjuvants, thickeners, excipients, etc. as described herein which is applied to a person's skin.

The term “skin” means the keratinous surfaces skin, hair and nails. The term “skin” when used herein is in the broad sense meaning the skin of the face, body, and neck as well as the lips.

In one aspect, the present invention provides a topical compositions comprising niacinamide mononucleotide and/or its derivative of formula (I) for improving the appearance of aging skin and/or treating or preventing skin aging. The compositions are expected to help address severe skin dryness, dullness, loss of elasticity, lack of radiance, exaggerated lines and wrinkles, spider vessels or red blotchiness. Particularly, marionette lines, smile lines, deep nasolabial fold lines, crow's feet, fine lines/wrinkles, vertical lines between the eyebrows, horizontal forehead lines, sagging thin/frail skin, skin redness and dullness may be improved using the compositions of the invention. When applied to skin, compositions of the present invention are expected to show improvement in surface spots, brown spots, red areas, wrinkles and texture and other artifacts of aging skin.

The term “surface spots” refers to brown or red spots which include freckles, acne marks or scars, hyperpigmentation and vascular lesions.

The term “brown spots” refers to those caused by an excess of melanin on and within the skin, these lesions include freckles, melasma, hyperpigmentation and lentigines.

The term “red areas” refers to various skin conditions such as acne, rosacea, inflammation and spider veins that have apparent red structures due to the blood vessels and hemoglobin contained in the papillary dermis.

The term “wrinkles” refers to fine lines, furrows, folds and creases in the skin. Wrinkles are associated with decreased skin elasticity.

The term “texture” refers to gradations in the skin's color and tone and surface peaks and valleys that are analyzed to measure smoothness.

Formula (I) has the following structure:

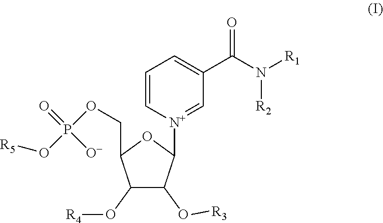

wherein each of R₁ and R₂ is individually selected from the group consisting of hydrogen, alkyl, cycloalkyl, alkenyl, alkaryl, hydroxy, alkoxy, and amino; each of R₃ and R₄ is individually selected from the group consisting of hydrogen, alkyl, aliphatic or aromatic acyl, and aliphatic or aromatic thioacyl; and R₅ is hydrogen, cation, or alkyl. When R₅ is hydrogen, the formula (I) may be in an ionic salt form when a sufficient amount of base is present (not shown). For example, the phosphate acid may be in a sodium salt form, wherein the covalent bond “O—R₅” may be replaced by an ionic bond, “O⁻Na⁺”. In a preferred embodiment, R₁, R₂, R₃, and R₄ are hydrogen, and R₅ is a sodium cation or hydrogen.

“Alkyl” refers to an alkyl group and substituted alkyl group wherein the alkyl group preferably has from 1 to about 12 carbon atoms, more preferably 1 to 8 carbon atoms and still more preferably 1 to 6 carbon atoms, wherein the alkyl group may be substituted with to 1 to 3 substituents selected from the group consisting of alkoxy, amino, mono- and dialkylamino, aminoacyl, aminocarbonyl, alkoxycarbonyl, aryl, carboxyl, cyano, halo, heterocyclic, hydroxy, nitro, thioalkoxy and the like.

“Cycloalkyl” refers to cyclic alkyl groups of from 3 to 10 carbon atoms having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantanyl, and the like.

“Alkenyl” refers to alkenyl groups preferably having from 2 to 10 carbon atoms and more preferably 2 to 6 carbon atoms and having at least 1 and preferably from 1-2 sites of alkenyl unsaturation. Preferred alkenyl groups include ethenyl, n-propenyl, isopropenyl, and the like.

“Alkaryl” refers to alkylene-aryl groups preferably having from 1 to 10 carbon atoms in the alkylene moiety and from 6 to 14 carbon atoms in the aryl moiety. Such alkaryl groups are exemplified by benzyl, phenethyl, and the like.

“Alkoxy” refers to the group “alkyl-O—”. Preferred alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Amino” refers to primary, secondary and tertiary alkyl substituted amino groups and the like.

“Acyl” refers to a monovalent group with a carbon atom of a carbonyl group as the point of attachment, further having a linear or branched, cyclo, cyclic or acyclic structure which may contain a heteroatom.

“Thioacyl” refers to a monovalent group with a carbon atom of a thiocarbonyl group as the point of attachment, further having a linear or branched, cyclo, cyclic or acyclic structure which may contain a heteroatom.

Nicotinamide mononucleotide is a naturally occurring compound in the body that plays a vital role in how cells use energy. Samarista-Giron, A. reported in 2011 that nicotinamide mononucleotide is able to reduce high blood sugar levels and elevated levels of cholesterol, triglycerides and free fatty acids during in vitro animal studies by with mice. Samhita, L. reported in 2013 that nicotinamide mononucleotide may rejuvenate mice's muscle tissues via intramuscular injection of nicotinamide mononucleotide. U.S. Application Publication No. 20160022712 to Imai et al. discloses the use of nicotinamide mononucleotide compositions for treating aged-related diseases and conditions, such as age-related obesity, age-related increases in blood lipid levels, age-related decreases in insulin sensitivity, age-related decreases in memory function, and age-related changes in eye function such as macular degeneration. U.S. Application Publication No. 20160024527 to Tilly et al. discloses the use of nicotinamide mononucleotide compositions for improving female fertility.

However, there is no disclosure in the art regarding topical administration of nicotinamide mononucleotide for anti-aging skin treatment or improving the appearance of aging skin. In particular, none of the prior art discloses suitable formulations that are sufficiently stable, have good bioavailability via topical administration on an affected skin, and effectively lead to anti-aging effects.

Niacinamide mononucleotide may be purchased commercially from Sigma-Aldrich. Nacinamide mononucleotide and its derivative compounds may also be prepared by various means known to those of skill in the art. For example, Leder I. G. discloses an in vitro synthesis of nitcotinamide mononucleotide. See Lder I. G., “Synthesis of nitcotinamide mononucleotide by human erythrocytes in vitro”, J. Biol. Chem. 1951, 189:889-899.

The compositions may further comprise a dermatologically acceptable carrier, and particularly one in which the niacinamide mononucleotide or its derivative is soluble per se or is effectively solubilized (e.g., as an emulsion or microemulsion). If a dermatologically acceptable carrier is employed, the carrier should be inert in the sense of not bringing about a deactivation or oxidation of the glutathione derived active ingredient(s), and in the sense of not bringing about any adverse effect on the skin areas to which it is applied.

In one preferred practice of the invention, one or more niacinamide mononucleotides and/or their derivatives are applied in admixture with the dermatologically acceptable carrier or vehicle (e.g., a solution, a dispersion, a lotion, a cream, an ointment, a soap, a solid stick, a gel, and the like) so as to facilitate topical application. In some cases, the carrier may provide additional therapeutic effects as might be brought about, e.g., by moisturizing of the affected skin areas. While the carrier for the topical composition can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration and/or aid in the percutaneous delivery of the active agent(s).

Many preparations are known in the art, and include lotions containing oils and/or alcohols and emollients vegetable oils, hydrocarbon oils and waxes, silicone oils, animal or marine fats or oils, glyceride derivatives, fatty acids or fatty acid esters, or alcohols (e.g., ethanol, propanediol, etc.) or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. In the preferred embodiment, the carrier is an oil-in-water emulsion. It is noticed that an oil-in-water emulsion system stabilizes the active ingredient therein. Without wishing to be bound by theory, the niacinamide mononucleotide and/or its derivatives have hydrophilic and hydrophoblic moieties in each molecule, which are more compatible and thus more stable in an oil-in-water emulsion system. Moreover, depending on the substituents of R₁ to R₅, the overall hydrophilicity of niacinamide mononucleotide and/or its derivatives may cover a large spectrum. An oil-in-water emulsion system is suitable for dissolving and stabilizing this class of active ingredients.

The quantity of niacinamide mononucleotide or its derivative active ingredient in the carrier may be varied or adjusted widely depending upon the particular application, the potency of the particular compound or the desired concentration. Generally, the quantity of niacinamide mononucleotide or its derivative active ingredient will range between about 0.05% to about 30% by weight, more preferably, about 0.1% to about 20.0% by weight. In some embodiments, niacinamide mononucleotide is present in an amount of about 0.1% to about 5.0% by weight. In further preferred embodiments, niacinamide mononucleotide is present from about 0.5% to about 1.5% by weight. Generally, lower concentrations of niacinamide mononucleotide or its derivative active ingredients in a carrier are suitable, depending upon the application regimen and the active and adjunct active ingredients employed. The term “by weight” shall mean by weight of the total composition, unless otherwise specified.

In some embodiments, the compositions of this invention contain at least one other active adjunct ingredient in addition to niacinamide mononucleotide or its derivative. The active adjunct ingredients may present in an amount ranging from 0.01% to about 20% by weight of the composition. They include, but are not limited to one or more of: isothiocyanates, caffeine, vitamin D3, lipoic acid; α-hydroxy acids such as glycolic acid or lactic acid; ascorbic acid and its derivatives, especially fatty acid esters of ascorbic acid; polyenylphosphatidylcholine; or tocotrienols and tocotrienol derivatives and vitamin E compositions enriched with tocotrienols or tocotrienol derivatives (e.g., tocotrienol acetate, also known as vitamin E acetate); and neuropeptides. Preferred adjunct agents include glycolic acid, citric acid, ascorbyl palmitate, a Sepitonic™ M3 by Seppic product (which contains magnesium aspartate, zinc gluconate and copper gluconate), a Tocomin® 50 product (which comprises palm oil, tocotrienols, tocopherol), and Oligopeptide-17 (which is a synthetic 35 amino acid peptide consisting of alanine, arginine, asparagine, aspartic acid, glutamine, glutamic acid, glycine, isoleucine, leucine, lysine, threonine and proline), and Oligopeptide-49 (which is a synthetic 35 amino acid peptide consisting of alanine, arginine, asparagine, aspartic acid, glutamine, glutamic acid, glycine, isoleucine, lysine, threonine, and proline).

The topical composition of the invention can contain additional ingredients commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition.

Emollients, typically present in amounts ranging from about 0.01% to about 20% of the total composition include, but are not limited to, fatty esters, fatty alcohols, mineral oils, polyether siloxane copolymers, docosahexanoic acid (DHA) and mixtures thereof. Preferred emollients are Actiglow® (hydrolyzed glycosaminoglycans, propylene glycol, water, phenoxethanol) by Active Organics, CCTG (carpric caprylic triglyceride), squalane, shae butter, meadowfoam seed oil, IPP (isopropyl palmitate), and DHA.

Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, L-tyrosine, and mixtures thereof. A preferred humectant is shae butter.

Emulsifiers, which are also called emulsifying agents, typically present in amounts from about 0.1% to about 70% by weight of the composition, include, but are not limited to, stearic acid, cetyl alcohol (as known as C-95 alcohol, or Crodacol™ C95, as sold by Croda Inc.), stearyl alcohol, steareth 2, steareth 20, acrylates/C10-30 alkyl acrylate cross polymers, silicones, dimethyl ethanolamine (DMAE), phosphatidylcholine (PPC), docosahexanoic acid (DHA) and mixtures thereof. Preferred emulsifiers are sodium hyaluronate, Promulgen-D® (a mixture of 75% cetostearyl alcohol and 25% ethoxylate cetostearyl alcohol sold by Amerchol Corp.), Polawax NF (cetostearyl alcohol and polysorbate 60 sold by a company called Coop Coco), Arlacel™ 165 (glyceryl stearate and PEG-100 Stearate sold by Croda Inc.), Crodesta™ F10 (sucrose distearate sold by Croda Inc.), silicone (Dow Corning® 200 Fluid, 350 CST), dimethylaminoethanol, also known as DMAE, and Phospholipon® 90 G (phosphatidylcholine with 0.1% ascorbyl palmitate sold by Phospholipid GmbH). Noticeably, some ingredients can have multiple functions. For examples, some ingredients, such as PPC can be both an emulsifier as well as an active adjunct ingredient.

One preferred emulsifier is PPC. By “polyenylphosphatidylcholine (PPC)” it meant any phosphatidylcholine (PC) bearing two fatty acid substituents, wherein at least one is an unsaturated fatty acid with at least two double bonds. In some embodiments, dilinoleoylphosphatidylcholine is the most abundant phosphatidylcholine species in polyenylphosphatidylcholine. Preferred PPCs contain at least one linoleic (18:2) group, most preferably two, in a cis geometrical configuration typical of natural products, for example, dilinoleoylphosphatidylcholine, which presents in the preparation at levels of at least about 25%, preferably at least about 40% by weight. Other forms of PPC can also be used as those set out in U.S. Pat. No. 6,797,459 at column 3 lines 34 to 52. PPC itself is an active antioxidant that has been shown to protect against lipid peroxidation and liver damage, including fibrosis and cirrhosis. Moreover, because PC itself is a major constituent of cell membranes, PPC greatly enhances the antioxidant activity of the composition because it facilitates the niacinamide nucleotide to penetrate and disperse in cell membranes in quantities sufficient to reach therapeutic levels.

Chelating agents, typically present in amounts ranging from about 0.01% to about 2% by weight, include, but are not limited to, ethylenediamine tetraacetic acid (EDTA) and derivatives and salts (e.g., sodium salt) thereof, dihydroxyethyl glycine, tartaric acid, and mixtures thereof.

Antioxidants, typically present in an amount ranging from about 0.01% to about 0.75% by weight of the composition, include, but are not limited to, butylated hydroxy toluene (BHT); vitamin C and/or vitamin C derivatives, such as fatty acid esters of ascorbic acid, particularly ascorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives (such as tocotrienol acetate, also known as vitamin E acetate); calcium pantothenates; green tea extracts; mixed polyphenols; and mixtures of any of these. Particularly preferred antioxidants are those that provide additional benefits to the skin such as ascorbyl palm itate, sesame seed oil, alpha-lipoic acid, and Tocomin® 50 (which comprises palm oil, tocotrienols, tocopherol).

Preservatives include, but are not limited to, C₁-C₃ alkyl parabens and phenoxyenthanol (e.g., benzyl alcohol), typically present in an amount ranging from about 0.1% to about 2.0% by weight percent, based on the total composition. A preferred preservative is ISP's Optiphen™ Plus, a liquid preservative formulation featuring a blend of phenoxyethanol, sorbic acid, and an emollient base.

Buffering agents are employed in many compositions. Preferably, the amount of buffering agent is one that results in compositions having a pH ranging from about 4.0 to about 8.5, more preferably from about 4.5 to about 7.0, most preferably from about 5.0 to about 6.0. Typical buffering agents are chemically and physically stable agents commonly found in cosmetics, and can include compounds that are also adjunct ingredients such as citric acid, malic acid, and glycolic acid buffers.

NMN and its derivatives are stable in the compositions of the present invention. No significant degradation, precipitation, or uneven distribution of the ingredients has been observed at room temperature. It is believed that the formulations as described above stabilize NMN and its derivatives and the adjunct active ingredients. Depending on the substituents of R₁ to R₅, the overall hydrophilicity of niacinamide mononucleotide and/or its derivatives may cover a large spectrum, from hydrophilic to hydrophobic. Likewise, the adjunct active ingredients also range from hydrophilic to hydrophobic. The formulations of the present invention are compatible to and thus stabilize this type of the actives and adjunct actives.

In some embodiments, the compositions comprise of about 0.1% to about 5.0% by weight, preferably about 0.1% to about 5.0% by weight, and even more preferably about 0.5% to about 1.5% by weight of niacinamide mononucleotide and its derivative of formula (I), about 2.0% to about 15% by weight of at least one emulsifier, and water. In some of these embodiments, the at least one emulsifier is PPC.

In some other embodiments, the compositions comprises about 1.0% to about 3.0%, preferably about 0.1% to about 3.0% by weight, and even more preferably about 0.5% to about 1.5% by weight of niacinamide mononucleotide and its derivative of formula (I), about 1.5% to about 5.0% by weight of at least one fatty acid derivative of stearic acid, about 1% to 5% by weight an emollient, and water. In some of these embodiments, the emollient is isopropyl palmitate (“IPP”).

In another aspect, the present invention provides a method of treating aging skin by topically applying the compositions containing niacinamide mononucleotide or its derivative to an affected skin. After treatment for the recommended period of time, decreased inflammation, irritation, and erythema of the skin are observed, along with an increased skin elasticity and suppleness. Particularly, marionette lines, smile lines, deep nasolabial fold lines, crow's feet, fine lines/wrinkles, vertical lines between the eyebrows, horizontal forehead lines, sagging thin/frail skin, skin redness and dullness are reduced.

Only effective amounts of topical compositions containing niacinamide mononucleotide or its derivative are needed to achieve the aforementioned benefits and prevent typical menopausal and aging effects on the skin. One advantage of the present invention is the use of very small amount of the active ingredient topically to achieve the anti-aging effect.

In the practice of methods of the invention, the topical composition is topically applied to the skin areas, such as that of the face, at predetermined intervals often as a moisturizer, lotion, or cream, it generally being the case that gradual improvement is noted with each successive application. Although immediate effects can be observed, enhanced results are observed when the topical composition is applied twice daily, preferably in the morning and evening, for an extended period of time. Insofar as has been determined based upon clinical studies to date, no adverse side effects are encountered. The composition may also be applied three, four, or more times a day.

In a further aspect, the present invention provides methods to improve the appearance of skin, prevent and treat skin aging, dryness, dullness, loss of elasticity and lack of radiance. Particularly, the present invention may be used to prevent or retard the appearance of spider vessels or red blotchiness associated with menopausal skin. In another embodiment, the present invention may be used to prevent or treat exaggerated lines and wrinkles. It is an advantage of the invention that compositions of the invention do not require a pharmaceutical prescription.

While not wishing to be bound by any theory, it is believed that niacinamide mononucleotide and/or derivatives composition, when administrated into a human body, increases intracellular levels of the two codehydrogenases, nicotinamide adenine dinucleotide (NAD) and nicotinamide adenine dinucleotide phosphate (NADP) in a body, which in turn activate a protein called SIRT1 to invigorate mitochondria inside living cells and inhibit NADPH, thereby improving metabolism throughout the body. It is further believed that as a result of the improved metabolism, the skin cell generation rate speeds up, leading to improvement of the appearance of wrinkles and fine lines, dryness, dullness or lack of radiance of skin, sagging, discoloration, or redness and blotchiness of skin. It is also proposed that niacinamide mononucleotide and/or derivatives exert antioxidant properties; thus, it may scavenge reactive oxygen species which is the key etiology of skin aging such as wrinkles, sag, poor texture, hyperpigmentation, and skin yellowness.

It is known that NAD and NADP have the following structures:

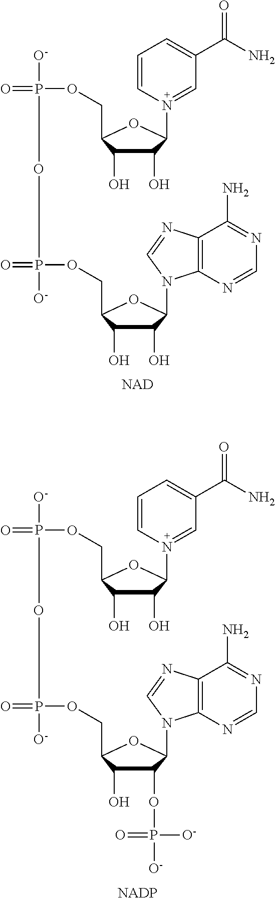

Compared to niacinamide or its derivatives known in the art, niacinamide mononucleotide contains a phosphate functional group on its molecules, which advantageously makes it more prone to be converted to NAD or NADP. The phosphate functional group may contribute to the high efficiency of niacinamide mononucleotide in increasing intracellular levels of NDA and NADP, thereby improving the anti-aging effects observed after topical administration of niacinamide mononucleotide on an affected skin.

Additional ingredients and methods as disclosed in Inventor's U.S. Pat. Nos. 5,376,361; 5,409,693; 5,545,398; 5,554,647; 5,574,063; 5,643,586; 5,709,868; 5,879,690; 6,191,121; 6,296,861; 6,437,004; 6,979,459; 8,609,604; 8,609,618, 9,023,801; and 9,029,317 which are hereby incorporated by reference, may also be used.

### EXAMPLES

### Example 1

A cream formulation in accordance with the present invention is shown below:

| Component | Amount (% w/w) |
|---|---|
| NMN | 0.05 to 1.5 |
| Sepitonic M3 | 0.5 to 5.0 |
| Vitamin E Acetate | 0.5 to 3.0 |
| Emulsifier(s) | 4.5 to 20.0 |
| Emolliment(s) | 5.0 to 15.0 |
| Chelating agent(s) | 0.05 to 0.5 |
| Preservative(s) | 0.5 to 3.0 |
| Water | q.s. to 100 |

### Example 2

A gel cream formulation in accordance with the present invention is shown below:

| Component | Amount (% w/w) |
|---|---|
| NMN | 10.0 to 25.0 |
| A second alcohol | 1.0 to 5.0 |
| Gel matrix | 70.0 to 89.0 |

Wherein the gel matrix has the following formulation:

| Component | Amount (% w/w) |
|---|---|
| Emulsifier(s) | 55.0 to 75.0 |
| Emolliment(s) | 16.0 to 36.0 |
| A first alcohol | 4.0 to 14.0 |

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention.

1. A topical composition for topical application to skin for prevention and treatment of skin aging, comprising:
from about 0.05% to about 30% by weight of β-niacinamide mononucleotide and/or its derivative of formula (I): 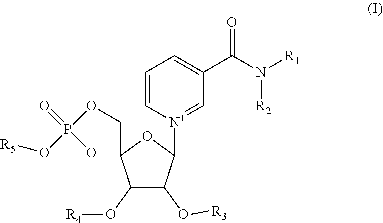
wherein each of R₁ and R₂ is individually selected from the group consisting of hydrogen, alkyl, cycloalkyl, alkenyl, alkaryl, hydroxy, alkoxy, and amino; each of R₃ and R₄ is individually selected from the group consisting of hydrogen, alkyl, aliphatic or aromatic acyl, and aliphatic or aromatic thioacyl; and R₅ is hydrogen, cation, or alkyl; and
a dermatologically acceptable carrier.

2. The method according to claim 1, wherein R₁ to R₄ are hydrogen, and wherein R₅ is a sodium cation or hydrogen.

3. The topical composition of claim 1, wherein the composition comprises: about 0.1% to about 5% by weight of niacinamide mononucleotide and its derivative of formula (I), about 2.0% to about 15% by weight of at least one emulsifier, and water.

4. The topical composition of claim 1, wherein the composition comprises: about 10.0% to about 30.0% by weight of niacinamide mononucleotide and its derivative of formula (I), about 30.0% to about 65.0% by weight of at least one emulsifier.

5. The topical composition of claim 1, wherein the emulsifier is a polyenylphosphatidylcholine.

6. A method for the prevention and treatment of skin aging comprising:
topically applying to affected skin areas a topical composition comprising:
β-niacinamide mononucleotide and/or its derivative of formula (I): 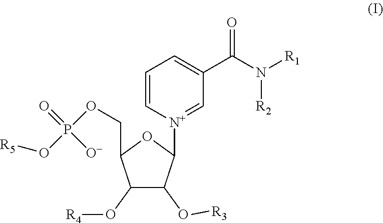
wherein each of R₁ and R₂ is selected from the group consisting of hydrogen, alkyl, cycloalkyl, alkenyl, alkaryl, hydroxy, alkoxy, and amino; each of R₃ and R₄ is selected from the group consisting of hydrogen, alkyl, aliphatic or aromatic acyl, and aliphatic or aromatic thioacyl; and R₅ is hydrogen, alkyl, or a cation; and
a dermatologically acceptable carrier.

7. The method according to claim 6, wherein R₁, R₂, R₃, and R₄ are hydrogen, and wherein R₅ is a sodium cation or hydrogen.

8. The method according to claim 6, wherein said topical composition contains from about 0.05% to about 30% of said β-niacinamide mononucleotide and/or its derivative of formula (I) by weight of the composition.

9. The method according to claim 8, wherein said topical composition contains from about 15.0% to about 20.0% of said β-niacinamide mononucleotide and/or its derivative of formula (I) by weight of the composition.

10. The method according to claim 8, wherein said topical composition contains from about 0.5% to about 1.5% of said β-niacinamide mononucleotide and/or its derivative of formula (I) by weight of the composition.

11. The method according to claim 6, wherein said carrier is a solution, dispersion, cream, lotion, gel, or solid stick.

12. The method according to claim 6, wherein the affected skin areas are skin areas having surface spots, brown spots, red areas, wrinkles, texture, skin dryness, dullness, loss of elasticity, lack of radiance, exaggerated lines and wrinkles, spider vessels, and/or red blotchiness.

13. The method according to claim 6, wherein said topical composition further comprises an adjunct ingredient selecting from the group consisting of isothiocyanates, caffeine, vitamin D3, lipoic acid, α-hydroxy acids, glycolic acid, lactic acid, ascorbic acid and its derivatives, fatty acid esters of ascorbic acid, polyenylphosphatidylcholine; tocotrienols, tocotrienol derivatives, vitamin E compositions enriched with tocotrienols or tocotrienol derivatives, neuropeptides, magnesium aspartate, zinc gluconate, copper gluconate, and a combination thereof.

14. The method according to claim 13, wherein said adjunct ingredient is in an amount of about 0.01% to about 20% by weight of the composition.

15. The method according to claim 13, wherein said adjunct ingredient is selected from the group consisting of lipoic acid, α-hydroxy acids, glycolic acid, lactic acid, ascorbic acid, ester of ascorbic acid, and a mixture thereof.

16. The method according to claim 13, wherein said adjunct ingredient is polyenylphosphatidylcholine.

17. The method according to claim 13, wherein said adjunct ingredient is selected from the group consisting of magnesium aspartate, zinc gluconate, copper gluconate, and a mixture thereof.

18. The method according to claim 6, wherein said topical composition further comprises an emollient in an amount ranging from about 0.01% to 20% of the composition.

19. The method according to claim 18, wherein said emollient is isopropyl palmitate, carpric caprylic triglyceride, or a combination thereof.

20. The method according to claim 6, wherein said topical composition has a pH range from about 4.0 to about 8.5.
